(19) United States
(12) Patent Application Publication
(10) Pub. No.: US 2022/0096437 A1
(43) Pub. Date: Mar. 31, 2022

(54) ANTIBACTERIAL COMPOUNDS

(71) Applicants: The University of Birmingham, Birmingham, West Midlands (GB); GUANGZHOU INSTITUTES OF BIOMEDICINE AND HEALTH, CHINESE ACADEMY OF SCIENCES, Guangzhou, Guangdong (CN)

(72) Inventors: Cleopatra Daniela NEAGOIE, Guangzhou, Guangdong (CN); Xudan PENG, Guangzhou, Guangdong (CN); Micky Daniel TORTORELLA, Guangzhou, Guangdong (CN); John S. FOSSEY, Birmingham, West Midlands (GB); Luke John ALDERWICK, Birmingham, West Midlands (GB); Antonia FEULA, San Mateo, CA (US); Akina YOSHIZAWA, Nagoya-shi, Aichi-ken (JP)

(21) Appl. No.: 17/602,426

(22) PCT Filed: Apr. 8, 2019
(86) PCT No.: PCT/CN2019/081767

§ 371 Date: Oct. 8, 2021

(51) Int. Cl. A61K 31/4025; A61K 45/06; A61K 31/397; A61K 31/4523

(52) U.S. Cl. CPC A61K 31/4025; A61K 31/4523; A61K 31/397; A61K 45/06

(57) ABSTRACT

Described are 1, 2, 4-substituted azetidine compounds of formula I, as well as pharmaceutical compositions and dosage forms comprising the compounds, and their use as a medicament. The compounds may find use as antibacterial agents, in particular against M. tuberculosis.

The present invention relates to azetidine compounds and their uses. In particular, the invention relates to 1,2,4-substituted azetidine compounds and their use as antibacterial agents.

Tuberculosis (TB) is an infectious disease caused by the bacterium Mycobacterium tuberculosis. In most healthy individuals the immune system is able to kill the bacteria. In some cases the immune system cannot kill the bacterial but controls its spread within the body. This is known as “latent” TB, which can develop into an active infection if the immune system becomes weakened.

TB is treated using antibiotics, which may be administered over a long duration. A typical treatment regimen for patients who have not previously had TB may last for six months, the first two months involving the administration of first line drugs such as isoniazid, pyrazinaminde and ethambutol, followed by continuation of isoniazid and rifampicin for the remaining four months.

Tuberculosis (TB) remains a major global health issue, despite it being over twenty years since the World Health Organisation (WHO) declared TB a global emergency. In 2016, TB killed approximately 1.3 million people and now ranks alongside HIV as the leading cause of death globally. It has been estimated that almost 6.3 million new cases of TB occurred in 2016; 46% of these new TB cases were individuals co-infected with HIV. Alarmingly, an estimated 4.1% of new TB cases and 19% of previously treated TB cases are infections caused by Multi-Drug Resistant TB (MDR-TB), and in 2016 an estimated 190,000 people died from this form of the disease. Furthermore, extensively drug-resistant TB (XDR-TB) has now been reported in 105 countries, and accounts for approximately 30,000 TB patients in 2016. If these numbers are to reduce in line with milestones set by the WHO End TB Strategy, alternative therapeutic agents that target novel pathways are urgently required.

The present invention has been devised with these issues in mind.

According to a first aspect of the present invention, there is provided a compound of formula I

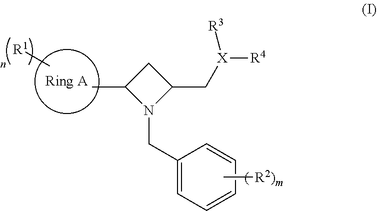

wherein:
ring A is a 6-membered ring, optionally containing at least one heteroatom;
each R¹ is independently selected from: halogen (e.g. fluorine, chlorine, bromine or iodine);
CZ₃, —OCZ₃, substituted or unsubstituted C₁₋₆ alkyl, alkenyl or alkynyl; OH, NO₂, CN, CHO, and CO₂R₅;
each R² is independently selected from: halogen (e.g. fluorine, chlorine, bromine or iodine); —CZ₃, —OCZ₃, substituted or unsubstituted C₁₋₆ alkyl, alkenyl or alkynyl; OH, NO₂, CN, CHO, and CO₂R₅;
X is nitrogen, carbon, sulfur or oxygen;
each Z is independently selected from fluorine, chlorine, bromine and iodine;
R³ and R⁴ independently represent hydrogen, substituted or unsubstituted C₁₋₆ alkyl, alkenyl or alkynyl, a cycloalkyl or heterocyclic ring, or a —(CH₂)_{q}—O—(CH₂)_{q} group, or X, R³ and R⁴ taken together form a structure selected from:

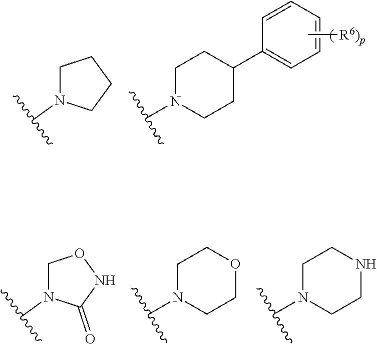

each R⁵ is independently selected from H, substituted or unsubstituted C₁₋₆ alkyl, benzyl, heteroaryl and aryl;
each R₆ is independently selected from —CZ₃ or OCZ₃,
n, m and p independently represent 0, 1, 2, 3, 4 or 5; and
each q is independently selected from any integer from 1 to 5, or a pharmaceutically acceptable salt thereof.

In some embodiments the compound is not a compound having a structure selected from:

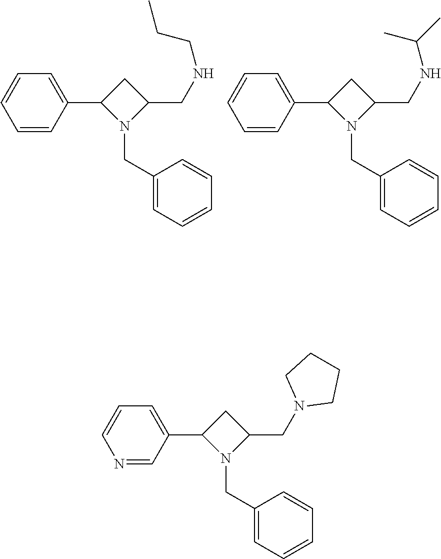

As used herein, the term “alkyl” refers to a straight- or branched-chain alkyl group. Examples of alkyl groups include methyl, ethyl, propyl, isopropyl, butyl, isobutyl, sec-butyl, tert-butyl, pentyl, isopentyl, tert-pentyl, hexyl, and isohexyl. Substituents may be attached at any point on the alkyl group.

The term “halogen” refers to fluorine, chlorine, bromine or iodine.

The term “cycloalkyl” refers to a saturated carbocycle (i.e. a ring formed of only carbon atoms) having from 3 to 7 ring atoms.

As used herein, the term “heterocyclic ring” refers to a monocyclic or fused bicyclic or tricyclic ring structure that has from 3 to 10 ring atoms per ring structure selected from carbon atoms and at least one (e.g. 1, 2, 3 or 4) heteroatom selected from nitrogen, oxygen and sulfur. The heterocyclic ring may be saturated or unsaturated. Examples of saturated heterocyclic rings include aziridine, oxirane, thiirane, azetidine, oxetane, thietane, pyrrolidine, tetrahydrofuran, tetrahydrothiophene, oxazolidine, dioxolane, dithiolane, piperidine, tetrahydropyran, and thiane. Examples of unsaturated heterocyclic rings include azirine, oxirene, thiirene, azete, oxete, thiete, pyrrole, furan, thiophene, pyridine, pyran, thiopyran and triazole.

The term “aryl” refers to an aromatic carbocycle (i.e. a ring or rings formed of only carbon atoms). The carbocycle may be monocyclic, or it may be a fused carbocycle. An aryl group may be substituted or unsubstituted. Examples of aryl groups include phenyl (C₆H₅) and naphthyl (C₁₀H₈).

The term “heteroaryl” refers to an aromatic ring or fused rings comprising at least one heteroatom (i.e. a non-carbon atom). The heteroatom may be selected from nitrogen, oxygen and sulfur.

The term “benzyl” refers to a phenyl ring which attached to the rest of the molecule by a methylene (CH₂) group. A benzyl group may be substituted or unsubstituted.

In some embodiments, Ring A is a heterocyclic ring.

In some embodiments Ring A is aromatic. In some embodiments, Ring A is phenyl.

In some embodiments, the aromatic ring contains one or more heteroatoms, such as nitrogen and/or oxygen. Thus, the ring may be a heterocyclic aromatic ring. For example, Ring A may be a pyridyl ring. In embodiments in which Ring A is a pyridine ring, the nitrogen atom may be arranged in the meta (3) position, relative to the bond linking the ring to the rest of the molecule.

Without wishing to be bound by theory, it is thought that electron withdrawing and/or lipophilic groups may be beneficial as R¹ and/or R².

In some embodiments each R¹ is independently selected from: halo, substituted or unsubstituted C₁₋₆ alkyl, —OZ₃, and —OCZ₃. The —OZ₃ group may be CF₃. The —OCZ₃ group may be OCF₃.

In further embodiments, each R¹ is independently selected from: Br, Cl, F, OCH₃, OCF₃, OH, CF₃ or t-butyl. In some embodiments each R¹ is independently selected from Br, F, OCF₃ and CF₃.

In some embodiments n is 1 or 2. In some embodiments wherein n is 1, the substituent R¹ may be in the ortho, meta or para position. In some embodiments wherein n is 2, the substituents may both be arranged in the meta positions.

In some embodiments, each R² is independently selected from: halo, substituted or unsubstituted C₁₋₆ alkyl, —CZ₃, and —OCZ₃. The —CZ₃ group may be CF₃. The —OCZ₃ group may be OC F₃.

In further embodiments, each R² is independently selected from: Br, Cl, F, OCH₃, Me (i.e. methyl, CH₃), OCF₃, OH, or CF₃. In some embodiments, each R² is independently selected from Br, F, OCH₃, Me, OCF₃ and CF₃.

In some embodiments m is 1 or 2. In some embodiments wherein m is 1, the substituent R² may be in the ortho, meta or para position. In some embodiments wherein m is 1, the substituent R² is in the ortho position.

In some embodiments, X is nitrogen. In some embodiments, R³ and R⁴ are independently selected from hydrogen and C₁₋₆ unsubstituted or substituted alkyl, such as methyl.

In some embodiments both R³ and R⁴ are methyl groups. Thus, in embodiments in which X is nitrogen, X, R³ and R⁴ together form a dimethylamine group.

In some embodiments X, R³ and R⁴ together form a pyrrolidine ring.

In some embodiments X, R³ and R⁴ together provide a structure selected from the following:

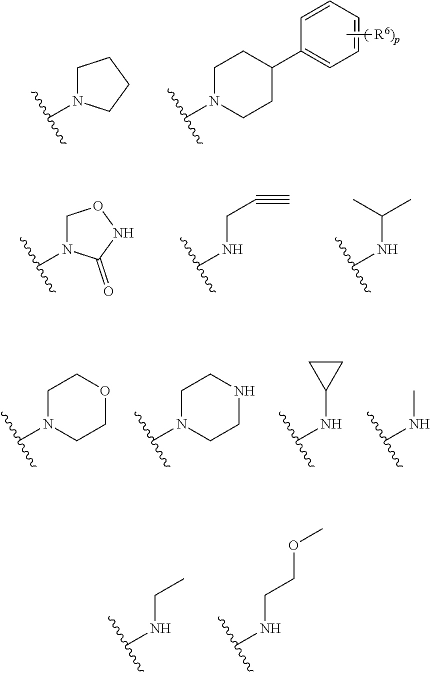

In some embodiments, p is 0 or 1. In some embodiments wherein p is 1, the substituent R⁶ is in the para position.

The compounds of the invention contain an azetidine ring. In particular, the compounds of the invention are 1,2,4-substituted azetidines.

The substituents in the 2- and 4-positions of the azetidine ring (the azetidine N being position 1) may be cis or trans. In some embodiments, the compounds are cis-azetidines.

The compound may be a racemic mixture, e.g. a racemic mixture of the 2,4-cis diasteroisomers. Alternatively, a single enantiomer may be provided.

In some embodiments, the compound has the structure according to formula II,

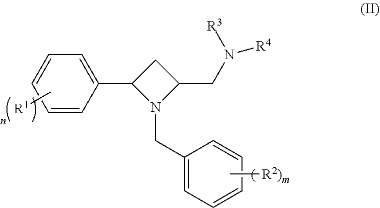

wherein
each R¹ is independently selected from Br, Cl, F, CF₃, OCF₃, OCH₃ and t-butyl,
each R² is independently selected from Br, Cl, F, CF₃, OCF₃, OCH₃ and CH₃, n and m independently represent 1 or 2;
R³ and R⁴ are independently selected from hydrogen and methyl, or N, R³ and R⁴ taken together form the structure:

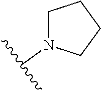 [SMILES: *N1CCCC1]

In some embodiments, each R¹ and R² is independently selected from Br, Cl, CF₃ and OCF₃.

In some embodiments, the compound according to the present invention is a compound selected from those listed in Table 1.

TABLE 1
Compounds according to the invention

| name | structure |
|---|---|
| Azet 1 | 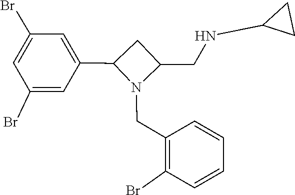 [SMILES: Brc1cc(Br)cc(C2CC(CNC3CC3)N2Cc2ccccc2Br)c1] |
| Azet 2 | 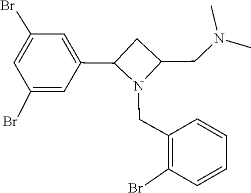 [SMILES: CN(C)CC1CC(c2cc(Br)cc(Br)c2)N1Cc1ccccc1Br] |
| Azet 3 | 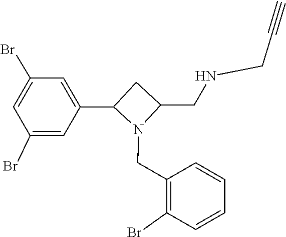 [SMILES: C#CCNCC1CC(c2cc(Br)cc(Br)c2)N1Cc1ccccc1Br] |
| Azet 4 | 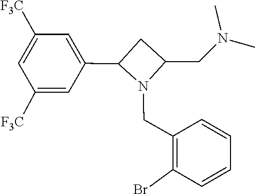 [SMILES: CN(C)CC1CC(c2cc(C(F)(F)F)cc(C(F)(F)F)c2)N1Cc1ccccc1Br] |
| Azet 5 | 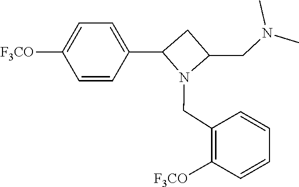 [SMILES: CN(C)CC1CC(c2ccc(OC(F)(F)F)cc2)N1Cc1ccccc1OC(F)(F)F] |
| Azet 6 | 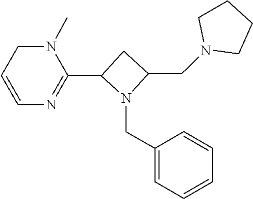 [SMILES: CN1CC=CN=C1C1CC(CN2CCCC2)N1Cc1ccccc1] |
| Azet 7 | 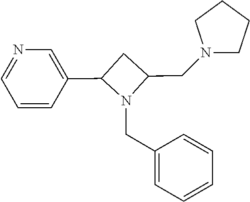 [SMILES: c1ccc(CN2C(CN3CCCC3)CC2c2cccnc2)cc1] |
| Azet 8 | 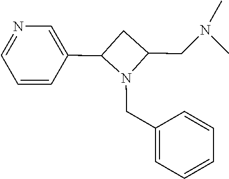 [SMILES: CN(C)CC1CC(c2cccnc2)N1Cc1ccccc1] |
| Azet 9 | 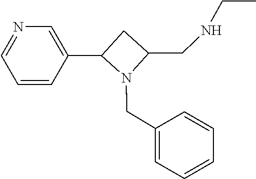 [SMILES: CCNCC1CC(c2cccnc2)N1Cc1ccccc1] |
| Azet 10 | 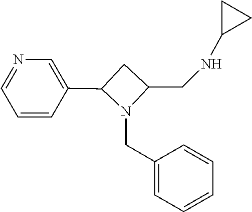 [SMILES: c1ccc(CN2C(CNC3CC3)CC2c2cccnc2)cc1] |
| Azet 11 | 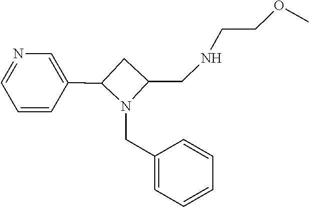 [SMILES: COCCNCC1CC(c2cccnc2)N1Cc1ccccc1] |
| Azet 12 | 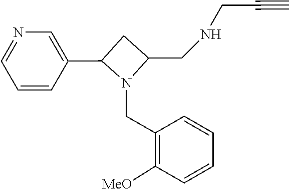 [SMILES: C#CCNCC1CC(c2cccnc2)N1Cc1ccccc1OC] |
| Azet 13 | 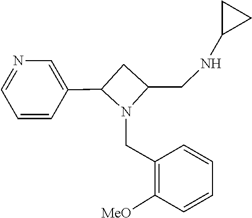 [SMILES: COc1ccccc1CN1C(CNC2CC2)CC1c1cccnc1] |
| Azet 14 | 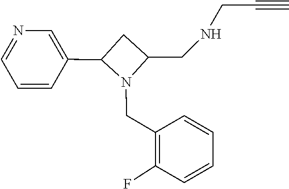 [SMILES: C#CCNCC1CC(c2cccnc2)N1Cc1ccccc1F] |
| Azet 15 | 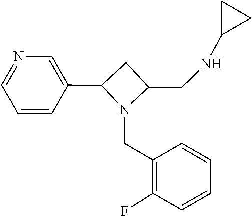 [SMILES: Fc1ccccc1CN1C(CNC2CC2)CC1c1cccnc1] |
| Azet 16 | 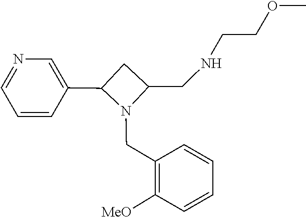 [SMILES: COCCNCC1CC(c2cccnc2)N1Cc1ccccc1OC] |
| Azet 17 | 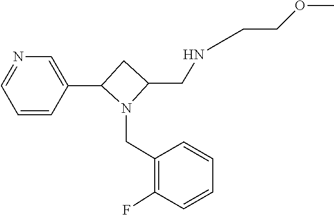 [SMILES: COCCNCC1CC(c2cccnc2)N1Cc1ccccc1F] |
| Azet 18 | 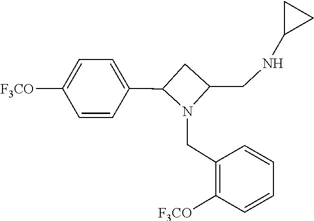 [SMILES: FC(F)(F)Oc1ccc(C2CC(CNC3CC3)N2Cc2ccccc2OC(F)(F)F)cc1] |
| Azet 19 | 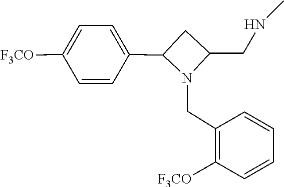 [SMILES: CNCC1CC(c2ccc(OC(F)(F)F)cc2)N1Cc1ccccc1OC(F)(F)F] |
| Azet 20 | 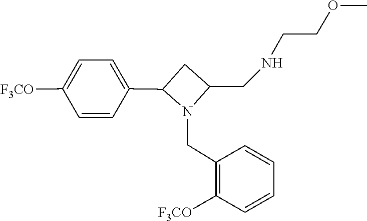 [SMILES: COCCNCC1CC(c2ccc(OC(F)(F)F)cc2)N1Cc1ccccc1OC(F)(F)F] |
| Azet 21 | 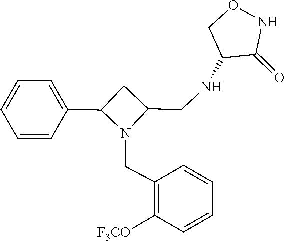 |
| Azet 22 | 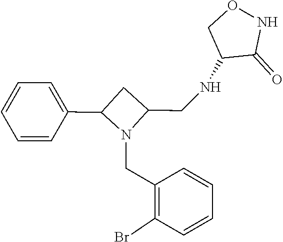 |
| Azet 23 | 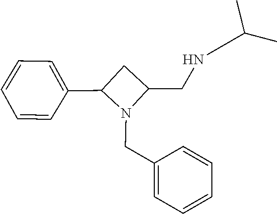 [SMILES: CC(C)NCC1CC(c2ccccc2)N1Cc1ccccc1] |
| Azet 24 | 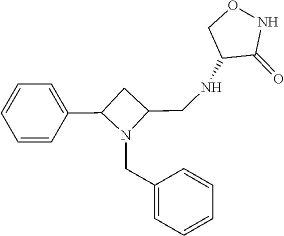 |
| Azet 25 | 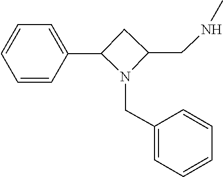 [SMILES: CNCC1CC(c2ccccc2)N1Cc1ccccc1] |
| Azet 26 | 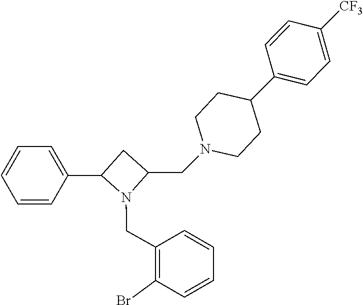 [SMILES: FC(F)(F)c1ccc(C2CCN(CC3CC(c4ccccc4)N3Cc3ccccc3Br)CC2)cc1] |
| Azet 27 | 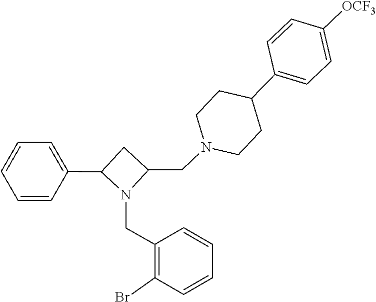 [SMILES: FC(F)(F)Oc1ccc(C2CCN(CC3CC(c4ccccc4)N3Cc3ccccc3Br)CC2)cc1] |
| Azet 28 | 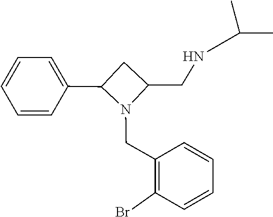 [SMILES: CC(C)NCC1CC(c2ccccc2)N1Cc1ccccc1Br] |
| Azet 29 | 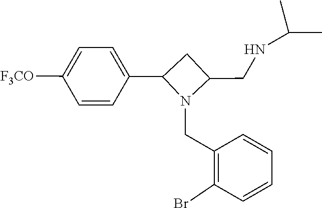 [SMILES: CC(C)NCC1CC(c2ccc(OC(F)(F)F)cc2)N1Cc1ccccc1Br] |
| Azet 30 | 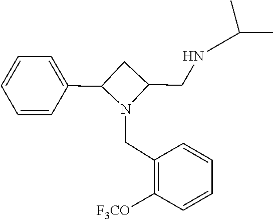 [SMILES: CC(C)NCC1CC(c2ccccc2)N1Cc1ccccc1OC(F)(F)F] |
| Azet 31 | 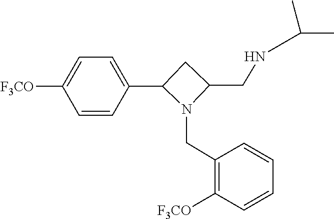 [SMILES: CC(C)NCC1CC(c2ccc(OC(F)(F)F)cc2)N1Cc1ccccc1OC(F)(F)F] |
| Azet 32 | 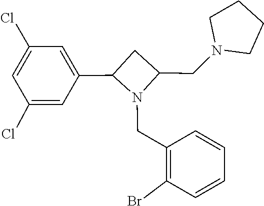 [SMILES: Clc1cc(Cl)cc(C2CC(CN3CCCC3)N2Cc2ccccc2Br)c1] |
| Azet 33 | 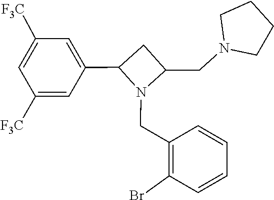 [SMILES: FC(F)(F)c1cc(C2CC(CN3CCCC3)N2Cc2ccccc2Br)cc(C(F)(F)F)c1] |
| Azet 34 | 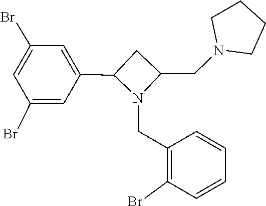 [SMILES: Brc1cc(Br)cc(C2CC(CN3CCCC3)N2Cc2ccccc2Br)c1] |
| Azet 35 | 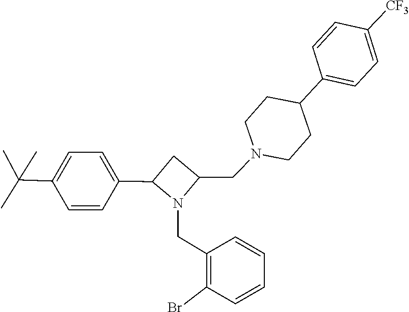 [SMILES: CC(C)(C)c1ccc(C2CC(CN3CCC(c4ccc(C(F)(F)F)cc4)CC3)N2Cc2ccccc2Br)cc1] |
| Azet 36 | 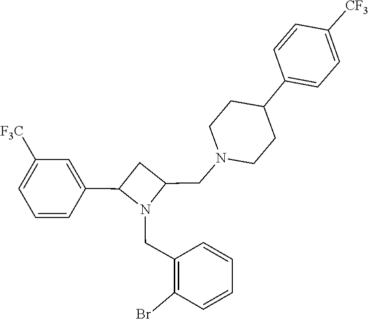 [SMILES: FC(F)(F)c1ccc(C2CCN(CC3CC(c4cccc(C(F)(F)F)c4)N3Cc3ccccc3Br)CC2)cc1] |
| Azet 37 | 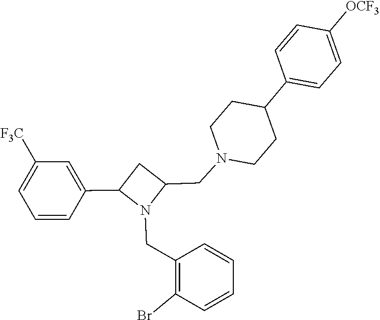 [SMILES: FC(F)(F)Oc1ccc(C2CCN(CC3CC(c4cccc(C(F)(F)F)c4)N3Cc3ccccc3Br)CC2)cc1] |
| Azet 38 | 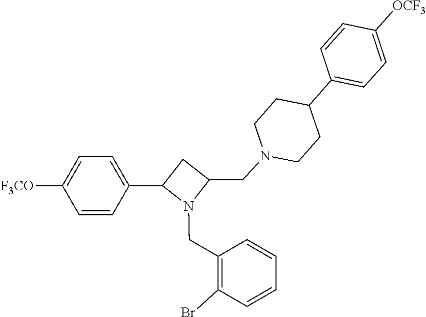 [SMILES: FC(F)(F)Oc1ccc(C2CCN(CC3CC(c4ccc(OC(F)(F)F)cc4)N3Cc3ccccc3Br)CC2)cc1] |
| Azet 39 | 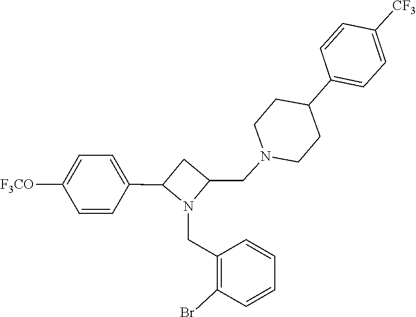 [SMILES: FC(F)(F)Oc1ccc(C2CC(CN3CCC(c4ccc(C(F)(F)F)cc4)CC3)N2Cc2ccccc2Br)cc1] |
| Azet 40 | 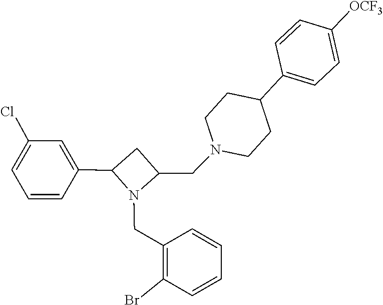 [SMILES: FC(F)(F)Oc1ccc(C2CCN(CC3CC(c4cccc(Cl)c4)N3Cc3ccccc3Br)CC2)cc1] |
| Azet 41 | 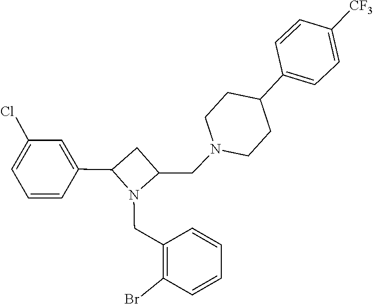 [SMILES: FC(F)(F)c1ccc(C2CCN(CC3CC(c4cccc(Cl)c4)N3Cc3ccccc3Br)CC2)cc1] |
| Azet 42 | 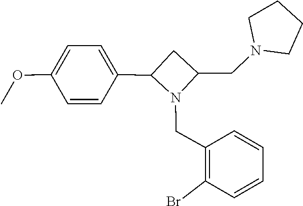 [SMILES: COc1ccc(C2CC(CN3CCCC3)N2Cc2ccccc2Br)cc1] |
| Azet 43 | 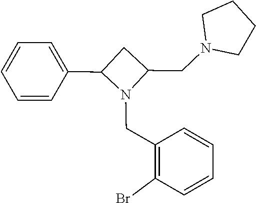 [SMILES: Brc1ccccc1CN1C(CN2CCCC2)CC1c1ccccc1] |
| Azet 44 | 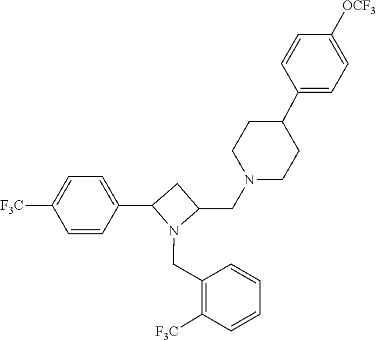 [SMILES: FC(F)(F)Oc1ccc(C2CCN(CC3CC(c4ccc(C(F)(F)F)cc4)N3Cc3ccccc3C(F)(F)F)CC2)cc1] |
| Azet 45 | 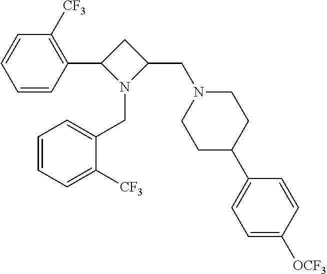 [SMILES: FC(F)(F)Oc1ccc(C2CCN(CC3CC(c4ccccc4C(F)(F)F)N3Cc3ccccc3C(F)(F)F)CC2)cc1] |
| Azet 46 | 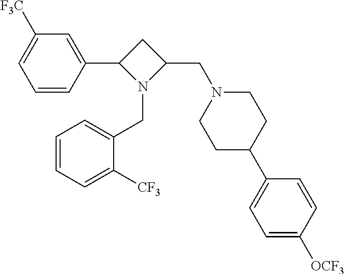 [SMILES: FC(F)(F)Oc1ccc(C2CCN(CC3CC(c4cccc(C(F)(F)F)c4)N3Cc3ccccc3C(F)(F)F)CC2)cc1] |
| Azet 47 | 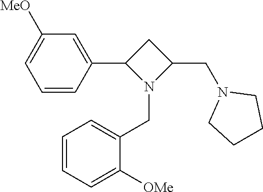 [SMILES: COc1cccc(C2CC(CN3CCCC3)N2Cc2ccccc2OC)c1] |
| Azet 48 | 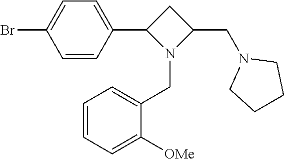 [SMILES: COc1ccccc1CN1C(CN2CCCC2)CC1c1ccc(Br)cc1] |
| Azet 49 | 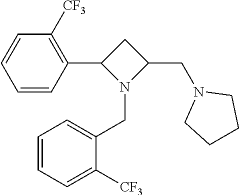 [SMILES: FC(F)(F)c1ccccc1CN1C(CN2CCCC2)CC1c1ccccc1C(F)(F)F] |
| Azet 50 | 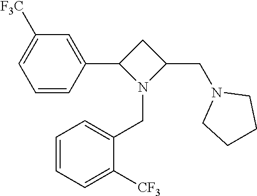 [SMILES: FC(F)(F)c1cccc(C2CC(CN3CCCC3)N2Cc2ccccc2C(F)(F)F)c1] |
| Azet 51 | 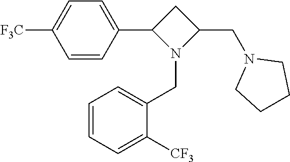 [SMILES: FC(F)(F)c1ccc(C2CC(CN3CCCC3)N2Cc2ccccc2C(F)(F)F)cc1] |
| Azet 52 | 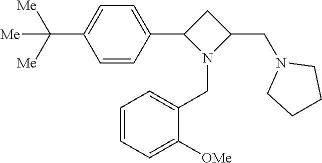 [SMILES: COc1ccccc1CN1C(CN2CCCC2)CC1c1ccc(C(C)(C)C)cc1] |
| Azet 53 | 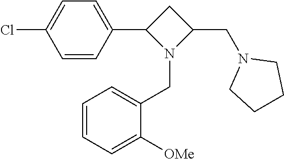 [SMILES: COc1ccccc1CN1C(CN2CCCC2)CC1c1ccc(Cl)cc1] |
| Azet 54 | 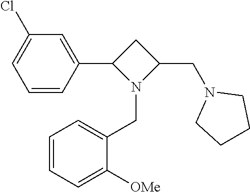 [SMILES: COc1ccccc1CN1C(CN2CCCC2)CC1c1cccc(Cl)c1] |
| Azet 55 | 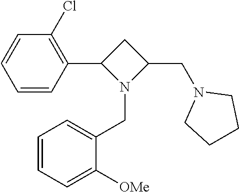 [SMILES: COc1ccccc1CN1C(CN2CCCC2)CC1c1ccccc1Cl] |
| Azet 56 | 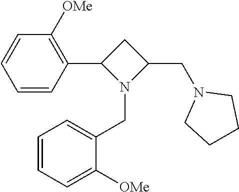 [SMILES: COc1ccccc1CN1C(CN2CCCC2)CC1c1ccccc1OC] |
| Azet 57 | 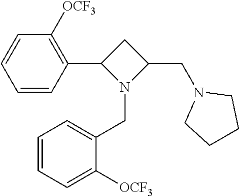 [SMILES: FC(F)(F)Oc1ccccc1CN1C(CN2CCCC2)CC1c1ccccc1OC(F)(F)F] |
| Azet 58 | 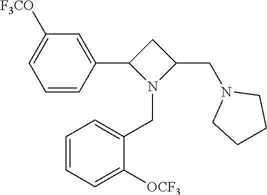 [SMILES: FC(F)(F)Oc1cccc(C2CC(CN3CCCC3)N2Cc2ccccc2OC(F)(F)F)c1] |
| Azet 59 | 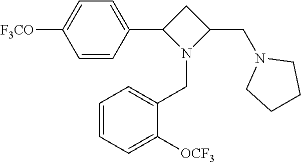 [SMILES: FC(F)(F)Oc1ccc(C2CC(CN3CCCC3)N2Cc2ccccc2OC(F)(F)F)cc1] |
| Azet 60 | 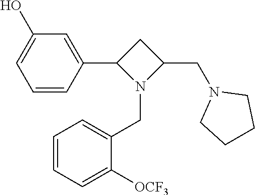 [SMILES: Oc1cccc(C2CC(CN3CCCC3)N2Cc2ccccc2OC(F)(F)F)c1] |
| Azet 61 | 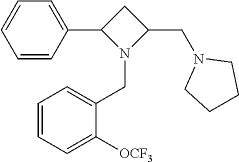 [SMILES: FC(F)(F)Oc1ccccc1CN1C(CN2CCCC2)CC1c1ccccc1] |
| Azet 62 | 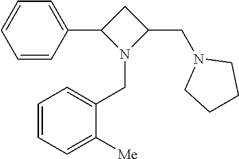 [SMILES: Cc1ccccc1CN1C(CN2CCCC2)CC1c1ccccc1] |
| Azet 63 | 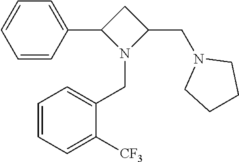 [SMILES: FC(F)(F)c1ccccc1CN1C(CN2CCCC2)CC1c1ccccc1] |
| Azet 64 | 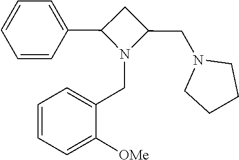 [SMILES: COc1ccccc1CN1C(CN2CCCC2)CC1c1ccccc1] |

It will be understood that different possible stereoisomers of the compounds described herein may be formed. In some embodiments the synthetic route may deliver a racemic mixture of the 2,4-cis diasteroisomers. The single enantiomers may then be obtained, for example through chiral auxiliary approaches to the allylation step and by chromatographic separation of the racemic azetidine derivatives using a chiral stationary phase in prep or semi-prep HPLC.

In some embodiments, the compound is a compound selected from Azet 2, Azet 4, Azet 5, Azet 19, Azet 32, Azet 33, Azet 34 and Azet 59 as shown in Table 1.

In some embodiments, the compound has a molecular weight of less than 700, less than 650, less than 600, less than 550 or less than 500.

In some embodiments, the compound has an MIC₅₀ and/or an MIC₉₉ against M. tuberculosis of less than 100 μM, less than 50 μM, less than 25 μM, less than 15 μM, less than 10 μM, less than 8 μM or less than 5 μM. In some embodiments the M. tuberculosis is M. tuberculosis strain H37Rv. Methods of determining MIC₉₉ and MIC₅₀ are described herein, and will be well-known to those skilled in the art.

The compounds of the invention may be in crystalline or amorphous form either as free compounds or as solvates (e.g. hydrates) and it is intended that all forms are within the scope of the present invention. Methods of solvation are generally known within the art.

According to a second aspect of the invention, there is provided a pharmaceutical composition comprising a compound as defined herein.

The pharmaceutical composition may comprise a therapeutically effective amount of the compound. The pharmaceutical composition may further comprise a pharmaceutically acceptable carrier or excipient.

According to a yet further aspect of the invention, there is provided a dosage form comprising a pharmaceutical composition according to the second aspect of the present invention.

In a yet further aspect of the invention, there is provided a compound as defined herein, a pharmaceutical composition or a dosage form according to the present invention for use as a medicament.

According to a further aspect of the present invention, there is provided a compound as defined herein or a pharmaceutically acceptable salt thereof, for use in the treatment of an infection.

The compound may be any compound as described herein.

In a further aspect of the invention, there is provided the use of a compound as defined herein in the manufacture of a medicament for the treatment of an infection.

In another aspect, the invention provides a method of treating a patient suffering from or at risk of an infection, the method comprising administering to the patient a therapeutically effective amount of a compound, or a pharmaceutical composition or dosage form according to the present invention.

The patient may be a mammal, in particular a human.

Pharmaceutical compositions of the invention can be formulated so as to allow a compound according to the present invention to be bioavailable upon administration of the composition to an animal, preferably human. Compositions can take the form of one or more dosage units, where for example, a tablet can be a single dosage unit, and a container of a compound according to the present invention may contain the compound in liquid or in aerosol form and may hold a single or a plurality of dosage units.

The pharmaceutically acceptable carrier or vehicle can be particulate, so that the compositions are, for example, in tablet or powder form. The carrier(s) can be liquid, with the compositions being, for example, an oral syrup or injectable liquid. In addition, the carrier(s) can be gaseous, or liquid so as to provide an aerosol composition useful in, for example inhalatory administration. Powders may also be used for inhalation dosage forms. The term “carrier” refers to a diluent, adjuvant or excipient, with which the compound according to the present invention is administered. Such pharmaceutical carriers can be liquids, such as water and oils including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, disaccharides, and the like. In addition, auxiliary, stabilizing, thickening, lubricating and coloring agents can be used. In one embodiment, when administered to an animal, the compounds and compositions according to the present invention, and pharmaceutically acceptable carriers are sterile. Water is a preferred carrier when the compounds according to the present invention are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The present compositions, if desired, can also contain minor amounts of wetting or emulsifying agents, or pH buffering agents.

When intended for oral administration, the composition is preferably in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more for the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agent such as sucrose or saccharin; a flavoring agent such as peppermint, methyl salicylate or orange flavoring; and a coloring agent.

When the composition is in the form of a capsule (e.g. a gelatin capsule), it can contain, in addition to materials of the above type, a liquid carrier such as polyethylene glycol, cyclodextrins or a fatty oil.

The composition can be in the form of a liquid, e.g. an elixir, syrup, solution, emulsion or suspension. The liquid can be useful for oral administration or for delivery by injection. When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

Examples of the administration form include without limitation oral, topical, parenteral, sublingual, rectal, vaginal, ocular and intranasal. Parenteral administration includes subcutaneous injections, intravenous, intramuscular, intrasternal injection or infusion techniques.

The amount of the compound according to the present invention that is effective in the treatment of a particular disorder or condition will depend on the nature of the disorder or condition, and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgement of the practitioner and each patient's circumstances.

The compositions comprise an effective amount of a compound of the present invention such that a suitable dosage will be obtained. The correct dosage of the compounds will vary according to the particular formulation, the mode of application, and its particular site, host and the disease being treated, e.g. cancer and, if so, what type of tumor. Other factors like age, body weight, sex, diet, time of administration, rate of excretion, condition of the host, drug combinations, reaction sensitivities and severity of the disease should be taken into account. Administration can be carried out continuously or periodically within the maximum tolerated dose.

Typically, the amount is at least about 0.01% of a compound of the present invention, and may comprise at least 80%, by weight of the composition. When intended for oral administration, this amount can be varied to range from about 0.1% to about 80% by weight of the composition. Preferred oral compositions can comprise from about 4% to about 50% of the compound of the present invention by weight of the composition.

Preferred compositions of the present invention are prepared so that a parenteral dosage unit contains from about 0.01% to about 10% by weight of the compound of the present invention. More preferred parenteral dosage unit contains about 0.5% to about 5% by weight of the compound of the present invention.

For intravenous administration, the composition is suitable for doses from about 0.1 mg/kg to about 250 mg/kg of the animal's body weight, preferably from about 0.1 mg/kg and about 20 mg/kg of the animal's body weight, and more preferably from about 1 mg/kg to about 10 mg/kg of the animal's body weight.

The present compositions can take the form of solutions, suspensions, emulsions, tablets, pills, pellets, capsules, capsules containing liquids, powders, sustained-release formulations, suppositories, emulsions, aerosols, sprays, suspensions, or any other form suitable for use.

The pharmaceutical compositions can be prepared using methodology well known in the pharmaceutical art. For example, a composition intended to be administered by injection can be prepared by combining a compound of the present invention with water, or other physiologically suitable diluent, such as phosphate buffered saline, so as to form a solution. A surfactant can be added to facilitate the formation of a homogeneous solution or suspension.

The infection may be an infection by a bacteria, virus, fungus, archaea, parasite or yeast. In some embodiments, the infection is a bacterial infection.

In some embodiments, the bacterial infection is an infection by mycobacterium. Examples of mycobacterium which may be treated by the compounds of the invention are Mycobacterium tuberculosis, Mycobacterium bovis, Mycobacterium africanum, Mycobacterium africanum and Mycobacterium canetti. In addition, we also include the Non Tuberculosis Mycobacteria (NTM) organisms Mycobacterium abscessuss, Mycobacterium ulcerans, Mycobacterium leprae, Mycobacterium marinum and Mycobacterium avium as being targeted by the compounds of the invention.

In some embodiments, the present invention provides a compound, composition or dosage form as described herein for use in the treatment of tuberculosis.

A compound according to the invention may be administered in combination with one or more other active agents, such as antibacterial agents. Thus, also provided is a combination of a compound according to the present invention, and one or more other antibacterial agents.

In some embodiments, the other antibacterial agent is an anti-tuberculosis agent.

The compound according to the invention and the other antibacterial agent may be administered simultaneously, separately or sequentially.

It will be appreciated that any of the embodiments described herein in relation to the first aspect of the invention may be combined with any other aspect of the invention, unless otherwise stated.

Embodiments of the invention will now be described by way of example.

### EXAMPLE 1

### Synthetic Method

The general synthetic scheme contains four steps, as exemplified by scheme 1 below. The first step is an imination reaction between a substituted benzaldehyde and a substituted benzylamine. The homoallyl amine motif is readily accessible through allylation of imines, which is the second step. In the third step, iodine-mediated cyclization of homoallyl amines at room temperature delivered cis-2,4-azetidine through a 4-exo trig cyclization. (Isomerization of iodo-azetidines to cis-pyrrolidines could be achieved by heating, with complete stereocontrol). Further functionalization, amination reaction, was achieved through nucleophilic displacement of iodine to deliver substituted azetidines, aminoazetidine.

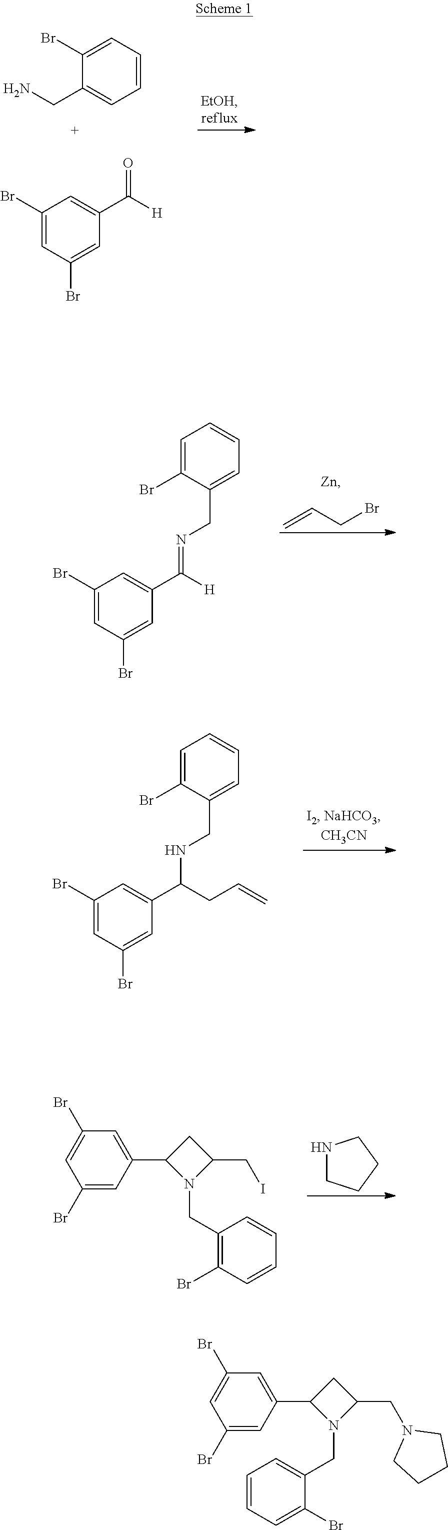

Imine formation: e.g. (i) EtOH, reflux (1 to 10 hr); or (ii) CH₂Cl₂ and dessicant (e.g. MS 3A); or (iii) Toluene, Δ, Dean Stark apparatus.
Allylation: e.g. (i) reaction with in situ prepared allyl since; or (ii) reaction with pre-formed allyl-magnesium (Grignard) reagents (typically RMgX, R=allyl, X=halogen).
Cyclisation: Typically with molecular iodine (I₂), an inorganic base (NaHCO₃), in a suitable solvent (acetonitrile) at temperatures not exceeding 25° C. (typically <18° C.), lower temperatures five cleaner conversion to desired azetidine-containing product but lead to extended reaction times (18 to 36 h may be required).
Amination: Addition of primary or secondary amine (or other nucleophile—e.g. azide) either neat (for liquid amines) or as a solution (required for solid amines) in a polar non-protic solvent (e.g. DMF or DMSO), displaces iodide and furnishes desired 1,2,3-azetidine derivatives.

All azetidines were made using the general protocol drawn in Scheme 1.

By well-established and routine procedures, aldehyde and amine were combined in solvent (EtOH) and heated at reflux for a period determined to be suitable as judged by TLC analysis (neutralized silica—EtOAc/hexane). Typically 1 to 10 hours to insure complete consumption of aldehyde. Solvent was evaporated and the residue thus obtained may be purified (if required as judged by inspection of the proton NMR spectrum of the residue thus obtained) by rapid filtration through a silica plug using EtOAc as eluent (removing any residual amine). By well-established and routine procedures, allylation was conducted by reaction of a preformed preferentially by addition an in situ prepared allylzinc reagent (activated zinc plus allyl bromide) in a suitable anhydrous solvent (e.g. THF (preferentially), dioxane or diethylether) (allyl Grignard or allyl stannane reagents also yield desired products zinc reagents provide cleaner products, less unwanted waste residues and smoother reactions). Purification of the homoallyl product thus obtained by flash chromatography (e.g. silica—EtOAc/hexane) typically improved the purity to >98% (by proton NMR spectroscopic analysis) thus permitting maximum yields in the next step. By procedures established within the teams of the co-inventors iodine mediated cyclisation was next conducted. Importantly, to avoid formation of unwanted (in this case) pyrrolidine products the reaction and subsequent product were held at <25° C., optimal conditions being 17° C. reaction, room temperature manipulation and storage at −4° C., furthermore the iodide containing products thus obtained were typically used immediately to avoid contamination by isomerization. Molecular iodine (I₂), an inorganic base (NaHCO₃), in a suitable solvent (acetonitrile) at temperatures not exceeding 25° C. (typically <18° C.), lower temperatures give cleaner conversion to desired azetidine-containing product but lead to extended reaction times (18 to 36 h may be required). Following aqueous work-up with sodium thiosulphate solution (to remove excess iodine), extraction, drying (over MgSO₄—or similar), filtration and solvent evaporation (at <20° C.) the residues thus obtained were analyzed by proton NMR spectroscopy to confirm the presence of azetidine derivatives (and absence of pyrrolidine derivatives by comparison to standard representative spectra) and used in the next step without further purification. To the iodo-azetidine derivatives thus obtained excess neat amine (if liquid) or amine dissolved in a suitable solvent (e.g. DMSO or DMF) as required. Following a period of stirring at room temperature or at temperatures controlled to be <18° C., depending on the ambient laboratory conditions temperature stabilization at 15° C. may be beneficial. Evaporation of volatiles and careful purification by flash chromatography (followed by preparative HPLC (C18 reverse phase) in some cases as desired) furnished analytically pure product 1,2,3-substituted azetidines.

### EXAMPLE 2

### Antibacterial Studies

Methodology

Strains

Mycobacterium tuberculosis H37Rv standard strain was tested by the proportion broth microdilution method. Bacteria were freshly grown on Middlebrook medium (7H9) supplemented with oleic acid, albumin, dextrose and catalase (OADC) enrichment.

Inoculum Preparation

Freshly grown colonies of M. tuberculosis from Middlebrook agar were transferred to a tube containing 3-4 ml phosphate buffered saline and 6 to 9 sterile glass beads. Tubes were vigorously agitated on a vortex mixer and clumps were allowed to settle for 30 min. The supernatants were transferred to sterile tubes. The supernatants were then adjusted with phosphate buffer saline to equal the density of 0.5 McFarland standard for use as the standard inoculum in the BMM and adjusted to equal the density of 1.0 McFarland for use as the standard inoculum for the proportion method (NCCLS 2002).

Compound Preparation

All compounds were prepared in a Greiner 96-well V-bottomed polypropylene microtitre plate (Compound Intermediate Plate). Compounds were loaded into column 2 at a concentration of 25 μM (100% DMSO) and 2-fold serially diluted in 100% DMSO from column 2 to 11 using a Hamilton Star robotics platform. Untreated control samples (column 1, 100% DMSO) and control compound (column 2, 10 mM Rifampicin) were included in microtitre plates.

In Vitro Anti-tubercular Activity Using a Selectable Marker-Free Autoluminescent Assay Against Mycobacterium tuberculosis H37Ra

UAIRa (Mtb H37Ra::pTYOK) was homogenized with sterile glass beads in a 50 ml tube containing 5 ml Middlebook 7H9 medium plus 0.05% Tween 80, 10% v/v oleic acid albumin dextrose catalase (OADC) supplement (7H9-OADC-Tw). When OD600 reached 0.3-0.5, relative light unit (RLU) count was determined by putting 200 μL culture on the detection hole of the luminometer. When the RLU reached 2 million, the effect concentration of compounds 5a-v was assessed over a range of 3-fold increasing from 0.000001 μg/mL to 10 μg/mL prepared in 25 μl UAIRa broth culture (RLU diluted to 2000-4000) grown in 7H9 broth with Tween80. In the treatment group, DMSO was used as negative control and Q203 (10 μg/mL, 1 μg/mL and 0.1 μg/L), isoniazide (INH, 10 μg/mL, 1 μg/mL and 0.1 μg/mL) and rifampicin (RIF, 10 μg/mL, 1 μg/mL and 0.1 μg/mL) were used as positive control. RLU 16 counts were determined daily, for 4 days. Analysis of the data, the MIClux value is the lowest drug concentration that can achieve the ratio (RLUdrug/RLUDMSO) less than 10% after treatment.

Susceptibility Testing Against Mycobacterium tuberculosis H37Rv

Greiner F-Bottom, black walled, clear bottom 96-well microtitre plates (assay plate) were filled with 100 μL Middlebrook 7H9 medium supplemented with oleic acid, albumin, dextrose and, catalase (OADC) enrichment. 1 μL of compounds were transferred from the Compound Intermediate Plate into the assay plate (including controls) using the 96-head of a Hamilton Star robotics platform. 100 μL of M. tuberculosis H37Rv in Middlebrook 7H9 supplemented with OADC was added to all 96-wells of the assay plate, this equates to approximately 5000 CFU per well of M. tuberculosis H37Rv per well. The assay plates were incubated at 37° C. for 7 days in a humidified incubator with 5% CO₂. On day 6 of incubation, 20 μL 0.02% resazurin was added to all 96-wells and plates were incubated for a further 24 hours at 37° C. On day 7 assay plates were measured fluorometrically using a BMG PheraSTAR FS with optic modules Exc—560 nm, Emm-585 nm. The percentage growth inhibition for each compound was calculated according to standard methods. MIC data was normalised against high and low controls and processed using Graphpad Prism software. Data was fitted to the Gomperz equation to determine MIC₉₉ values.

Results

Compounds were synthesized and tested according to the protocols above. The antibacterial activity of the compounds against M. tuberculosis H37Rv is shown in Table 2. The results show that azetidine compounds according to the invention are effective against M. tuberculosis.

TABLE 2
Antibacterial activity of compounds of the invention

| | | | | MIC₅₀ | MIC₉₉ |
|---|---|---|---|---|---|
| | | Mwt | | (μM) | (μM) |
| | | (g/ | | M. tub | M. tub |
| Ref. | Structure | mol) | Chemical Name | H37Rv | H37Rv |
| Azet 34 | 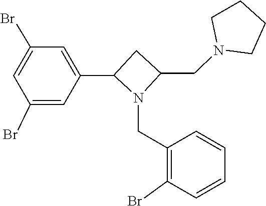 [SMILES: Brc1cc(Br)cc(C2CC(CN3CCCC3)N2Cc2ccccc2Br)c1] | 543.141 | rac-1-(((2,4-cis)-1-(2- bromobenzyl)-4-(3,5- dibromophenyl)azetidin- 2-yl)methyl)pyrrolidine | 7.03 | 3.26 |
| Azet 33 | 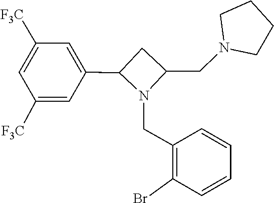 [SMILES: FC(F)(F)c1cc(C2CC(CN3CCCC3)N2Cc2ccccc2Br)cc(C(F)(F)F)c1] | 521.345 | rac-1-(((2,4-cis)-4-(3,5- bis(trifluoromethyl)phenyl)- 1-(2- bromobenzyl)azetidin-2- yl)methyl)pyrrolidine | 6.93 | 3.29 |
| Azet 59 | 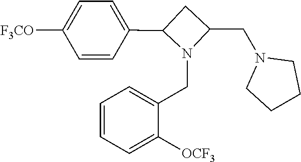 [SMILES: FC(F)(F)Oc1ccc(C2CC(CN3CCCC3)N2Cc2ccccc2OC(F)(F)F)cc1] | 474.447 | rac-1-(((2,4-cis)-1-(2- (trifluoromethoxy)benzyl)- 4-(4- (trifluoromethoxy)phenyl) azetidin-2- yl)methyl)pyrrolidine | 9.42 | 6.25 |
| Azet 19 | 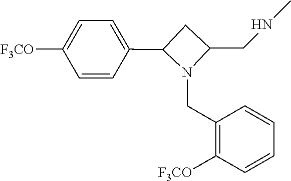 [SMILES: CNCC1CC(c2ccc(OC(F)(F)F)cc2)N1Cc1ccccc1OC(F)(F)F] | 434.382 | rac-N-methyl-1-((2,4-cis)- 1-(2- (trifluoromethoxy)benzyl) (trifluoromethoxy)phenyl) azetidin-2- yl)methanamine | 4.5 | 7.2 |
| Azet 4 | 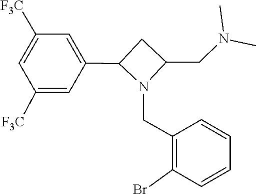 [SMILES: CN(C)CC1CC(c2cc(C(F)(F)F)cc(C(F)(F)F)c2)N1Cc1ccccc1Br] | 495.307 | rac-1-((2,4-cis)-4-(3,5- bis(trifluoromethyl)phenyl)- 1-(2- bromobenzyl)azetidin-2- yl)-N,N- dimethylmethanamine | 6.59 | 7.3 |
| Azet 32 | 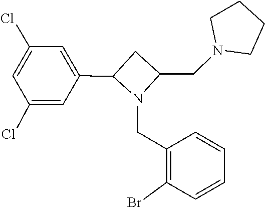 [SMILES: Clc1cc(Cl)cc(C2CC(CN3CCCC3)N2Cc2ccccc2Br)c1] | 454.23 | rac-1-(((2,4-cis)-1-(2- bromobenzyl)-4-(3,5- dichlorophenyl)azetidin- 2-yl)methyl)pyrrolidine | 7.58 | 9.21 |
| Azet 5 | 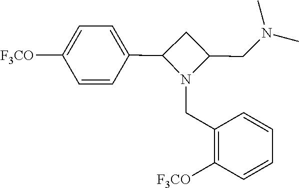 [SMILES: CN(C)CC1CC(c2ccc(OC(F)(F)F)cc2)N1Cc1ccccc1OC(F)(F)F] | 448.409 | rac-N,N-dimethyl-1-((2,4- cis)-1-(2- (trifluoromethoxy)benzyl)- 4-(4- (trifluoromethoxy)phenyl) azetidin-2- yl)methanamine | 9.9 | 12.0 |
| Azet 18 | 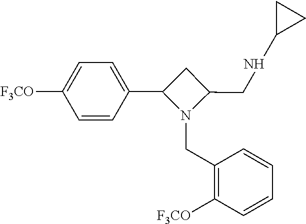 [SMILES: FC(F)(F)Oc1ccc(C2CC(CNC3CC3)N2Cc2ccccc2OC(F)(F)F)cc1] | 460.42 | rac-N-(((2,4-cis)-1-(2- (trifluoromethoxy)benzyl)- 4-(4- (trifluoromethoxy)phenyl) azetidin-2- yl)methyl)cyclopropana- mine | 11.9 | 13.0 |
| Azet 20 | 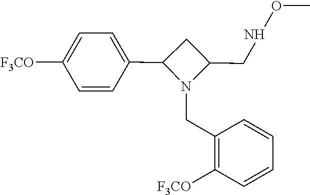 [SMILES: CONCC1CC(c2ccc(OC(F)(F)F)cc2)N1Cc1ccccc1OC(F)(F)F] | 478.435 | rac-2-methoxy-N-(((2,4- cis)-1-(2- (trifluoromethoxy)benzyl)- 4-(4- (trifluoromethoxy)phenyl) azetidin-2- yl)methyl)ethan-1-amine | 11.0 | 18.0 |
| Azet 29 | 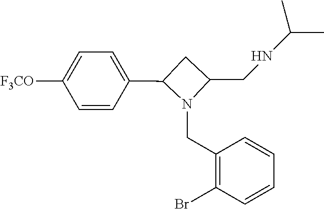 [SMILES: CC(C)NCC1CC(c2ccc(OC(F)(F)F)cc2)N1Cc1ccccc1Br] | 457.335 | rac-N-(((2,4-cis)-1-(2- bromobenzyl)-4-(4- (trifluoromethoxy)phenyl) azetidin-2- yl)methyl)propan-2- amine | 9.85 | 24.5 |
| Azet 31 | 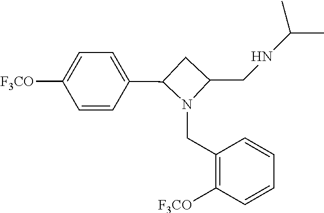 [SMILES: CC(C)NCC1CC(c2ccc(OC(F)(F)F)cc2)N1Cc1ccccc1OC(F)(F)F] | 462.436 | rac-N-(((2,4-cis)-1-(2- (trifluoromethoxy)benzyl)- 4-(4- (trifluoromethoxy)phenyl) azetidin-2- yl)methyl)propan-2- amine | 8.89 | 26.4 |
| Azet 42 | 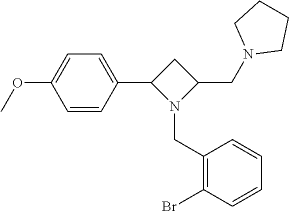 [SMILES: COc1ccc(C2CC(CN3CCCC3)N2Cc2ccccc2Br)cc1] | 415.375 | rac-1-(((2,4-cis)-1-(2- bromobenzyl)-4-(4- methoxyphenyl)azetidin- 2-yl)methyl)pyrrolidine | 20.8 | 42.6 |
| Azet 30 | 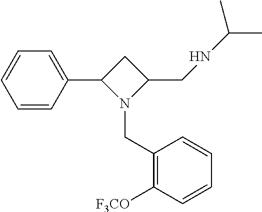 [SMILES: CC(C)NCC1CC(c2ccccc2)N1Cc1ccccc1OC(F)(F)F] | 378.439 | rac-N-(((2,4-cis)-4- phenyl-1-(2- (trifluoromethoxy)benzyl) azetidin-2- yl)methyl)propan-2- amine | 21.1 | 48.9 |
| Azet 1 | 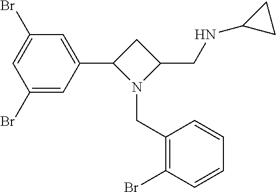 [SMILES: Brc1cc(Br)cc(C2CC(CNC3CC3)N2Cc2ccccc2Br)c1] | 529.114 | rac-N-(((2,4-cis)-1-(2- bromobenzyl)-4-(3,5- dibromophenyl)azetidin- 2- yl)methyl)cyclopropana- mine | 11.8 | 51.8 |
| Azet 39 | 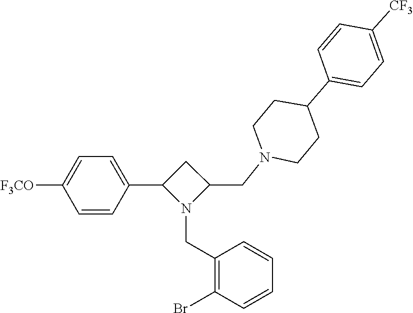 [SMILES: FC(F)(F)Oc1ccc(C2CC(CN3CCC(c4ccc(C(F)(F)F)cc4)CC3)N2Cc2ccccc2Br)cc1] | 627.469 | rac-1-(((2,4-cis)-1-(2- bromobenzyl)-4-(4- (trifluoromethoxy)phenyl) azetidin-2-yl)methyl)-4- (4- (trifluoromethyl)phenyl)pi- peridine | 85.0 | 90.9 |
| Azet 2 | 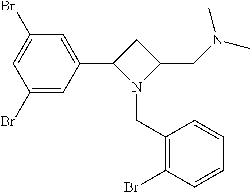 [SMILES: CN(C)CC1CC(c2cc(Br)cc(Br)c2)N1Cc1ccccc1Br] | 517.103 | rac-1-((2,4-cis)-1-(2- bromobenzyl)-4-(3,5- dibromophenyl)azetidin- 2-yl)-N,N- dimethylmethanamine | 5.6 | |
| Azet 53 | 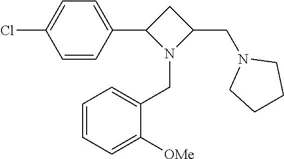 [SMILES: COc1ccccc1CN1C(CN2CCCC2)CC1c1ccc(Cl)cc1] | 370.92 | rac-1-(((2,4-cis)-4-(4- chlorophenyl)-1-(2- methoxybenzyl)azetidin- 2-yl)methyl)pyrrolidine | 10.3 | |
| Azet 52 | 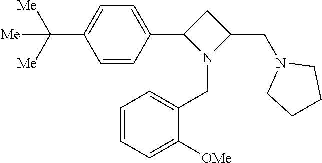 [SMILES: COc1ccccc1CN1C(CN2CCCC2)CC1c1ccc(C(C)(C)C)cc1] | 430.559 | rac-1-(((2,4-cis)-4-(4- (tert-butyl)phenyl)-1-(2- (trifluoromethyl)benzyl)a- zetidin-2- yl)methyl)pyrrolidine | 8.62 | |
| Azet 51 | 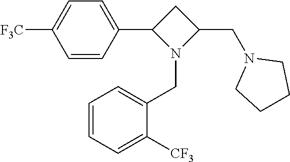 [SMILES: FC(F)(F)c1ccc(C2CC(CN3CCCC3)N2Cc2ccccc2C(F)(F)F)cc1] | 442.449 | rac-1-(((2,4-cis)-1-(2- (trifluoromethyl)benzyl)- 4-(4- (trifluoromethyl)phenyl)a- zetidin-2- yl)methyl)pyrrolidine | 8.31 | |
| Azet 50 | 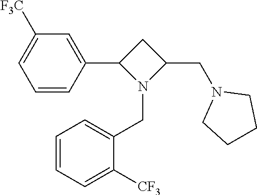 [SMILES: FC(F)(F)c1cccc(C2CC(CN3CCCC3)N2Cc2ccccc2C(F)(F)F)c1] | 442.449 | rac-1-(((2,4-cis)-1-(2- (trifluoromethyl)benzyl)- 4-(3- (trifluoromethyl)phenyl)a- zetidin-2- yl)methyl)pyrrolidine | 7.04 | |
| Azet 49 | 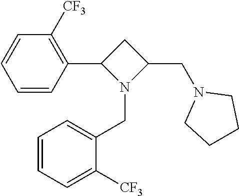 [SMILES: FC(F)(F)c1ccccc1CN1C(CN2CCCC2)CC1c1ccccc1C(F)(F)F] | 442.449 | rac-1-(((2,4-cis)-1-(2- (trifluoromethyl)benzyl)- 4-(2- (trifluoromethyl)phenyl)a- zetidin-2- yl)methyl)pyrrolidine | 6.68 | |
| Azet 48 | 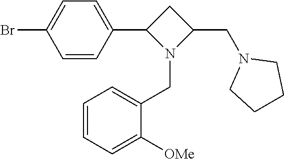 [SMILES: COc1ccccc1CN1C(CN2CCCC2)CC1c1ccc(Br)cc1] | 415.375 | rac-1-(((2,4-cis)-4-(4- bromophenyl)-1-(2- methoxybenzyl)azetidin- 2-yl)methyl)pyrrolidine | 20.3 | |
| Azet 63 | 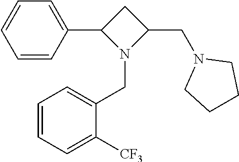 [SMILES: FC(F)(F)c1ccccc1CN1C(CN2CCCC2)CC1c1ccccc1] | 374.451 | rac-1-(((2,4-cis)-4- phenyl-1-(2- (trifluoromethyl)benzyl)a- zetidin-2- yl)methyl)pyrrolidine | 22.7 | |
| Azet 62 | 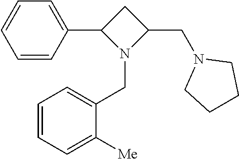 [SMILES: Cc1ccccc1CN1C(CN2CCCC2)CC1c1ccccc1] | 320.48 | rac-1-(((2,4-cis)-1-(2- methylbenzyl)-4- phenylazetidin-2- yl)methyl)pyrrolidine | 37.0 | |
| Azet 61 | 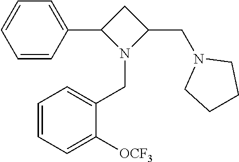 [SMILES: FC(F)(F)Oc1ccccc1CN1C(CN2CCCC2)CC1c1ccccc1] | 390.45 | rac-1-(((2,4-cis)-4- phenyl-1-(2- (trifluoromethoxy)benzyl) azetidin-2- yl)methyl)pyrrolidine | 17.9 | |
| Azet 58 | 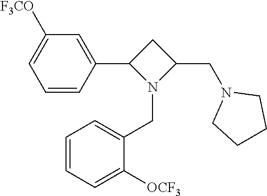 [SMILES: FC(F)(F)Oc1cccc(C2CC(CN3CCCC3)N2Cc2ccccc2OC(F)(F)F)c1] | 474.447 | rac-1-(((2,4-cis)-1-(2- (trifluoromethoxy)benzyl) (trifluoromethoxy)phenyl) azetidin-2- yl)methyl)pyrrolidine | 9.14 | |
| Azet 57 | 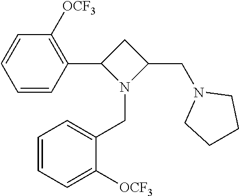 [SMILES: FC(F)(F)Oc1ccccc1CN1C(CN2CCCC2)CC1c1ccccc1OC(F)(F)F] | 474.447 | rac-1-(((2,4-cis)-1-(2- (trifluoromethoxy)benzyl) (trifluoromethoxy)phenyl) azetidin-2- yl)methyl)pyrrolidine | 7.05 | |
| Azet 55 | 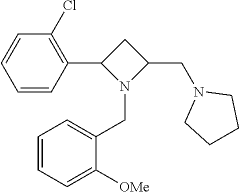 [SMILES: COc1ccccc1CN1C(CN2CCCC2)CC1c1ccccc1Cl] | 370.92 | rac-1-(((2,4-cis)-4-(2- chlorophenyl)-1-(2- methoxybenzyl)azetidin- 2-yl)methyl)pyrrolidine | 27.6 | |
| Azet 3 | 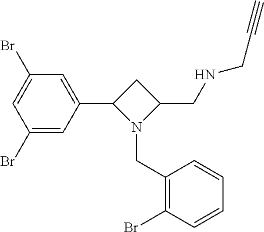 [SMILES: C#CCNCC1CC(c2cc(Br)cc(Br)c2)N1Cc1ccccc1Br] | 527.098 | rac-N-(((2,4-cis)-1-(2- bromobenzyl)-4-(3,5- dibromophenyl)azetidin- 2-yl)methyl)prop-2-yn-1- amine | 22.8 | |
| Azet 54 | 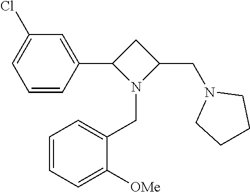 [SMILES: COc1ccccc1CN1C(CN2CCCC2)CC1c1cccc(Cl)c1] | 370.92 | rac-1-(((2,4-cis)-4-(3- chlorophenyl)-1-(2- methoxybenzyl)azetidin- 2-yl)methyl)pyrrolidine | 11.7 | |

1-19. (canceled)

20. A method of treating a patient in need, the method comprising administering to the patient a compound of formula I, 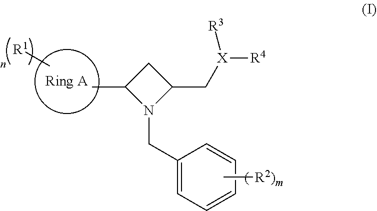
wherein:
ring A is a 6-membered ring, optionally containing at least one heteroatom;
each R¹ is independently selected from: halogen (e.g. fluorine, chlorine, bromine or iodine); —CZ₃, —OCZ₃, substituted or unsubstituted C₁₋₆ alkyl, alkenyl or alkynyl; OH, NO₂, CN, CHO, and CO₂R₅;
each R² is independently selected from: halogen (e.g. fluorine, chlorine, bromine or iodine); —CZ₃, —OCZ₃, substituted or unsubstituted C₁₋₆ alkyl, alkenyl or alkynyl; OH, NO₂, CN, CHO, and CO₂R₅;
X is nitrogen, carbon, sulfur or oxygen;
each Z is independently selected from fluorine, chlorine, bromine and iodine;
R³ and R⁴ independently represent hydrogen, substituted or unsubstituted C₁₋₆ alkyl, alkenyl or alkynyl, a C₃-C₆ cycloalkyl or heterocyclic ring, or a —(CH₂)_{q}—O—(CH₂)_{q} group, or X, R³ and R⁴ taken together form a structure selected from: 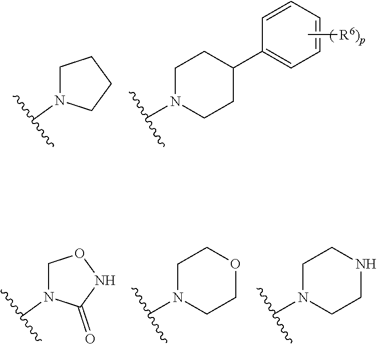
each R⁵ is independently selected from H, substituted or unsubstituted C₁₋₆ alkyl, benzyl, heteroaryl and aryl;
each R₆ is independently selected from —CZ₃ or OCZ₃,
n, m and p independently represent 0, 1, 2, 3, 4 or 5; and
each q is independently selected from any integer from 1 to 5.

21. The method according to claim 20, wherein Ring A is a phenyl or a pyridyl ring.

22. The method according to claim 20, wherein each R¹ is independently selected from: Br, Cl, F, OCH₃, OCF₃, OH, CF₃ or t-butyl.

23. The method according to claim 20, wherein each R² is independently selected from: Br, Cl, F, OCH₃, Me, OCF₃, OH, or CF₃.

24. The method according to claim 20, wherein n is 1 or 2.

25. The method according to claim 20, wherein m is 1 or 2.

26. The method according to claim 20, wherein R³ and R⁴ are independently selected from hydrogen and C₁₋₆ unsubstituted or substituted alkyl.

27. The method according to claim 20, wherein X is nitrogen.

28. The method according to claim 27, wherein X, R³ and R⁴ together form a pyrrolidine ring or a dimethyl amine group.

29. The method according to claim 20, wherein the compound is a cis-azetidine.

30. The method according to claim 20, wherein the compound has an MIC₅₀ and/or an MIC₉₉ against M. tuberculosis of less than 25 μM.

31. The method according to claim 20, in the treatment of an infection.

32. The method according to claim 31, wherein the infection is a bacterial infection.

33. The method according to claim 32, wherein the bacterial infection is an infection by mycobacterium.

34. The method according to claim 32, wherein the method comprises administering to the patient at least one other antibacterial agent.

35. A pharmaceutical composition comprising a compound of formula I, 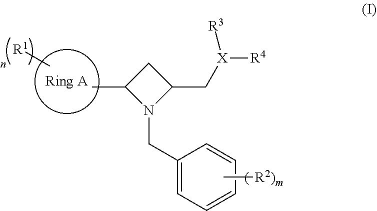
wherein:
ring A is a 6-membered ring, optionally containing at least one heteroatom;
each R¹ is independently selected from: halogen (e.g. fluorine, chlorine, bromine or iodine); —CZ₃, —OCZ₃, substituted or unsubstituted C₁₋₆ alkyl, alkenyl or alkynyl; OH, NO₂, CN, CHO, and CO₂R₅;
each R² is independently selected from: halogen (e.g. fluorine, chlorine, bromine or iodine); —CZ₃, —OCZ₃, substituted or unsubstituted C₁₋₆ alkyl, alkenyl or alkynyl; OH, NO₂, CN, CHO, and CO₂R₅;
X is nitrogen, carbon, sulfur or oxygen;
each Z is independently selected from fluorine, chlorine, bromine and iodine;
R³ and R⁴ independently represent hydrogen, substituted or unsubstituted C₁₋₆ alkyl, alkenyl or alkynyl, a C₃-C₆ cycloalkyl or heterocyclic ring, or a —(CH₂)_{q}—O—(CH₂)_{q} group, or X, R³ and R⁴ taken together form a structure selected from: 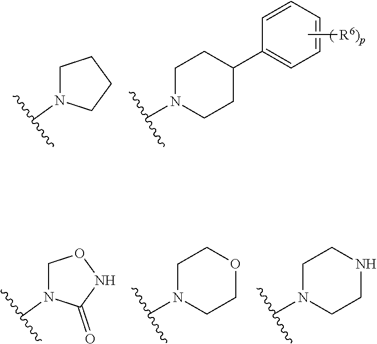
each R⁵ is independently selected from H, substituted or unsubstituted C₁₋₆ alkyl, benzyl, heteroaryl and aryl;
each R₆ is independently selected from —CZ₃ or OCZ₃,
n, m and p independently represent 0, 1, 2, 3, 4 or 5; and
each q is independently selected from any integer from 1 to 5.

36. A dosage form comprising a pharmaceutical composition according to claim 35.

37. A combination comprising a pharmaceutical formulation according to claim 35 and at least one other antibacterial agent.

38. A compound of formula I 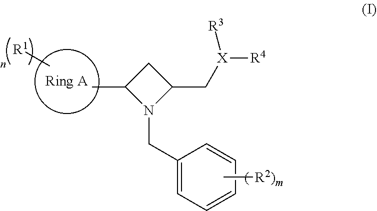
wherein:
ring A is a 6-membered ring, optionally containing at least one heteroatom;
each R¹ is independently selected from: halogen (e.g. fluorine, chlorine, bromine or iodine); —CZ₃, —OCZ₃, substituted or unsubstituted C₁₋₆ alkyl, alkenyl or alkynyl; OH, NO₂, CN, CHO, and CO₂R₅;
each R² is independently selected from: halogen (e.g. fluorine, chlorine, bromine or iodine); —CZ₃, —OCZ₃, substituted or unsubstituted C₁₋₆ alkyl, alkenyl or alkynyl; OH, NO₂, CN, CHO, and CO₂R₅;
X is nitrogen, carbon, sulfur or oxygen;
each Z is independently selected from fluorine, chlorine, bromine and iodine;
R³ and R⁴ independently represent hydrogen, substituted or unsubstituted C₁₋₆ alkyl, alkenyl or alkynyl, a C₃-C₆ cycloalkyl or heterocyclic ring, or a —(CH₂)_{q}—O—(CH₂)_{q} group, or X, R³ and R⁴ taken together form a structure selected from: 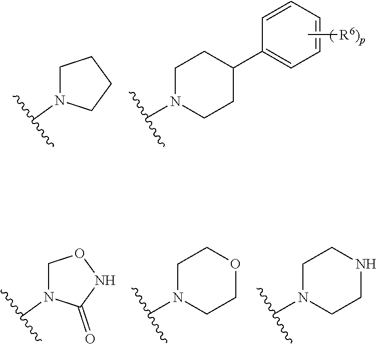
each R⁵ is independently selected from H, substituted or unsubstituted C₁₋₆ alkyl, benzyl, heteroaryl and aryl;
each R₆ is independently selected from —CZ₃ or OCZ₃,
n, m and p independently represent 0, 1, 2, 3, 4 or 5; and
each q is independently selected from any integer from 1 to 5, or a pharmaceutically acceptable salt thereof,
wherein the compound is not a compound having a structure selected from: [chemical structure]
